(19) United States
(12) Patent Application Publication
(10) Pub. No.: US 2026/0061198 A1
(43) Pub. Date: Mar. 5, 2026

(54) Sleep Apnea Adaptive Detection And Treatment System

(71) Applicant: Neurostim Technologies LLC, Wellesley, MA (US)

(72) Inventors: Hoo-min D. TOONG, Cambridge, MA (US); William C. ALTMANN, Austin, TX (US); Hans SCHELLENBERG, Redwood City, CA (US); Terry SPURLING, Menlo Park, CA (US); Anthony WEI, Palo Alto, CA (US); Luke ARCENEAUX, San Francisco, CA (US)

(21) Appl. No.: 19/314,332

(22) Filed: Aug. 29, 2025

Related U.S. Application Data
(60) Provisional application No. 63/689,140, filed on Aug. 30, 2024.
(60) Provisional application No. 63/689,146, filed on Aug. 30, 2024.
(60) Provisional application No. 63/689,153, filed on Aug. 30, 2024.
(60) Provisional application No. 63/689,162, filed on Aug. 30, 2024.
(60) Provisional application No. 63/699,255, filed on Sep. 26, 2024.
(60) Provisional application No. 63/714,293, filed on Oct. 31, 2024.

(51) Int. Cl. A61N 1/36; A61B 5/00; A61N 1/04

(52) U.S. Cl. CPC A61N 1/36034; A61B 5/4818; A61B 5/4836; A61B 5/4848; A61N 1/0456; A61N 1/0476; A61N 1/0492; A61N 1/3601; A61N 1/36031

(57) ABSTRACT

Embodiments treat obstructive sleep apnea (OSA) of a user. Embodiments affix a patch externally on a dermis of the user, the patch including a flexible substrate, a processor coupled to the substrate, and an array of electrodes coupled to the substrate, the array of electrodes including a plurality of electrode pairs in different geometric positions on the substrate. Embodiments detect an occurrence of OSA. In response to the detecting, embodiments generate a first electrical stimuli to one or more nerves or tissues of the user via a first selected subset of pairs of the plurality of electrode pairs. Embodiments generate a second electrical stimuli to one or more nerves or tissues of the user via a second selected subset of pairs of the plurality of electrode pairs sequentially after generating the first electrical stimuli, where the first selected subset of pairs are different than the second selected subset of pairs.

### CROSS REFERENCE TO RELATED APPLICATIONS

This application claims priority to U.S. Provisional Patent Application Ser. No. 63/689,140 filed on Aug. 30, 2024, U.S. Provisional Patent Application Ser. No. 63/689,146 filed on Aug. 30, 2024, U.S. Provisional Patent Application Ser. No. 63/689,153 filed on Aug. 30, 2024, U.S. Provisional Patent Application Ser. No. 63/689,162 filed on Aug. 30, 2024, U.S. Provisional Patent Application Ser. No. 63/699,255 filed on Sep. 26, 2024 and U.S. Provisional Patent Application Ser. No. 63/714,293 filed on Oct. 31, 2024. The disclosure of each of the applications is hereby incorporated by reference.

### FIELD

Embodiments are directed to systems and methods for improving sleep by detecting the patterns of obstructive sleep apnea episodes and/or treating each episode.

### BACKGROUND INFORMATION

Obstructive sleep apnea affects the quality of sleep. Obstructive Sleep Apnea (“OSA”) is the intermittent occlusion of the upper airway (“UAW”), resulting in the reduction of airflow through the throat. This may be due to neuromuscular factors or anatomical causes. The muscles that keep the airway open when active can allow it to close when relaxed. An obstructed airflow causes imbalances in oxygen exchange, measurable in the hemoglobin of the blood.

The equipment used for individuals suffering from OSA includes detection of each episode of OSA, in order to apply some treatment to the individual to reduce the effects of such an OSA episode. Detection of an episode occurs once the episode has begun, when the distress of interrupted breathing has begun.

In the context of sleep and sleep quality, current medical processes provide the subject with an index referred to as the “Apnea/Hypopnea Index”, or “AHI”, which is a count of the total number of events of any type of apnea or hypopnea per hour, as manually scored by a sleep professional and calculated during a sleep period. Apnea and hypopnea differ in their effect upon SpO2 and other body systems. By combining both into a single index, subjects with identical AHI numbers can have very different health outcomes. The AHI parameter provides little insight into the related biometrics that affect sleep disturbances, nor into related co-morbidities that affect the severity of a person's apnea or hypopnea.

Polysomnography (“PSG”) records the subject's sleep typically for only one night. Variations in sleep habits and sleep disturbances are therefore not entirely recorded when the study extends over one, two or only a few nights. Similarly, PSG does not typically record sleep on nights separated from one another, such as once-per-week or once-per-month, or long-term studies on subjects. This limitation also constrains such data gathering. PSG signals are used for more than AHI events such as sleep stages, REM sleep (EOG), arousals (EEG), SpO2 desaturation, etc.

PSG provides the clinician with a suite of measured parameters, usually recorded through the client's sleep period. The complexity and nuances in the data require a professional reading of the results to provide an adequate report in the sleep disturbances. Due to the lack of experienced professional resources, the reading of a PSG study is expensive, usually requires several days to deliver to the client, and provides little closed-loop discussion of the results.

PSG delivers data on sleep at the end of the recording session or later. This after-the-fact delivery does not provide the opportunity to affect the subject's sleep during the recording of sleep data or to compare sleep data in real time to other physiological metrics. PSG studies, whether at a clinic or at home, involve wearing uncomfortable or obstructive equipment. This limits their continued use by those who want or need to assess their sleep quality.

### BRIEF DESCRIPTION OF THE DRAWINGS

FIG. 1 is an Obstructive Sleep Apnea Detection and Stimulation System in accordance to embodiments.

FIGS. 2A-FIG. 2D are diagrams of OSA-Related Nerves and Structures.

FIG. 3 is a User and an Obstructive Sleep Apnea Detection and Stimulation System in accordance to embodiments.

FIG. 4 is an example Polysomnography Setup.

FIGS. 5A and 5B illustrate numbered electrodes arranged in a rectangular matrix as an example of a multi-electrode array on a patch in accordance to embodiments.

FIG. 6 shows a Nasal Device as an example shape and placement for a patch in accordance to embodiments.

FIG. 7 shows a Forehead Device as an example shape and placement for a patch in accordance to embodiments.

FIG. 8 shows a Facial Device as an example shape and placement for a patch in accordance to embodiments.

FIG. 9 shows a Submental Device as an example shape and placement for a patch in accordance to embodiments.

FIG. 10 shows a system with both a Nasal Device and a Submental Device as example shapes and placements for a patch in accordance to embodiments.

FIG. 11 illustrates an Apnea Detection and Stimulation System in accordance to embodiments.

FIG. 12 is a flow diagram of the functionality of the Obstructive Sleep Apnea Detection and Stimulation System when monitoring sleep while treating apnea events and modifying the hypoxia level in accordance to embodiments.

FIG. 13 is a block diagram of the elements of an Obstructive Sleep Apnea Detection and Stimulation System in accordance to embodiments.

Further embodiments, details, advantages, and modifications will become apparent from the following detailed description of the embodiments, which is to be taken in conjunction with the accompanying drawings.

### DETAILED DESCRIPTION

Embodiments are directed to detecting an OSA episode and/or to treating/counteracting the apnea using neurostimulation, without the complex system of a conventional sleep study in a clinic or obtrusive treatment devices such as a continuous positive airway pressure (“CPAP”) machine.

Embodiments are directed to an integrated system that is placed on the skin of the user and activated and used with or without the help of a medical professional. The integrated system includes hardware and software to monitor biometrics related to breathing, and optionally, a computing resource able to analyze the breathing data and send improved parametric values to the device on the user's skin to improve the detection capabilities for that specific user, in a closed-loop system.

Embodiments also may include a computing resource to send signals to circuitry in the device or devices on the user's skin to create electrical stimulation through one or more electrodes. Embodiments may include one or more sensors to monitor the effectiveness of the electrical stimulation and, through the computing resource, adapt the electrical stimulation by selecting a different subset from among the set of electrodes to perform the next stimulation. The timing, wave shape, amplitude and duration of the electrical stimulation may be adjusted by the computing resource such that the effectiveness of the stimulation is improved on the particular user with the particular placement of the device on the user's skin. This closed loop adaptation of stimulation through the use of multiple, selectable electrodes improves the effectiveness over a system with a fixed set of electrodes which cannot adapt to variations in use or placement.

Embodiments can predict an OSA episode in real time and then use electrical stimulation to prevent the episode from proceeding, thereby limiting it to less than ten seconds such that it does not count in the calculation of AHI. Importantly, shortened or eliminated apnea episodes reduce the load on the user's physiology by reducing hypoxic burden, by reducing the amount of sleep fragmentation from arousals, and reducing stress upon the sympathetic nervous system. The prediction of each apnea episode is confirmed by the embodiments using biometric measurements in parallel with the prediction and treatment process such that no sleep expert annotations are needed, the correlation can confirm apnea episodes as well as confirming the elimination of apnea episodes all in real time.

Example inventions acquire a subject's sleep data using a device configured as a flexible patch with sensors and computational capabilities, such as disclosed in U.S. Pat. No. 11,712,557, the disclosure of which is hereby incorporated by reference.

Reference will now be made in detail to the embodiments of the present disclosure, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, and circuits have not been described in detail so as not to unnecessarily obscure aspects of the embodiments. Wherever possible, like reference numbers will be used for like elements.

### Patch

A method and system is disclosed that physicians may offer to their patients to detect an OSA episode in preparation for the use of neurostimulation to counteract the apnea, without the complex system of a conventional sleep study in a clinic. The method and system create one or more signals which may be utilized by treatment equipment without the need for additional sensing subsystems.

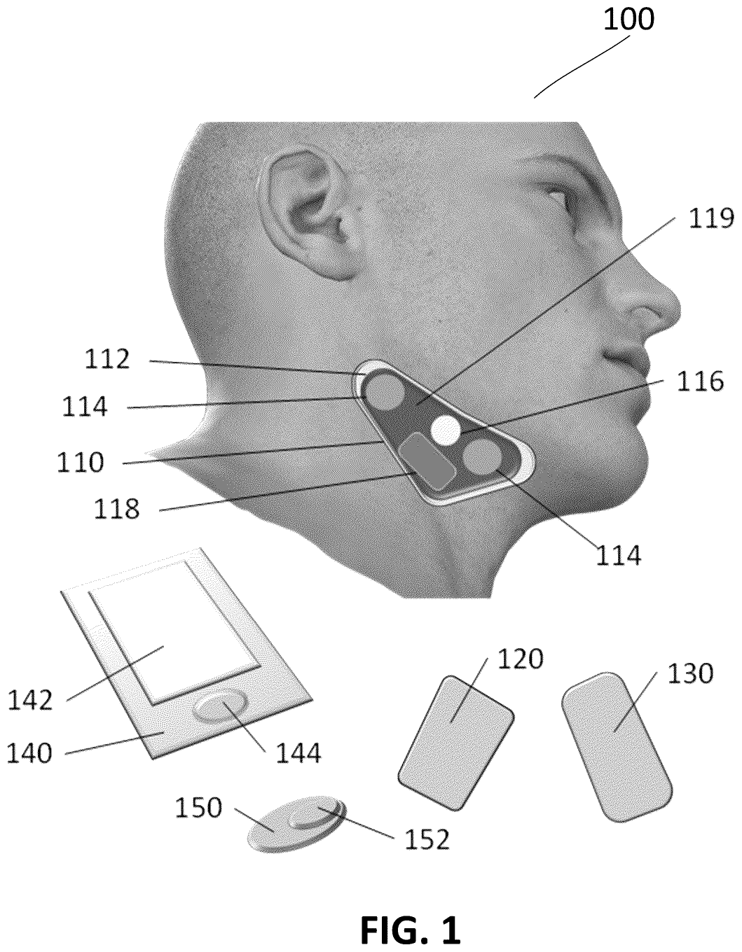

FIG. 1 is an illustration of components of an Obstructive Sleep Apnea Detection and Stimulation System 100, including a Neck Topical Nerve Activator (“TNA”) Device 110 (or “patch” 110), with a securing mechanism 112, and one or more electrode pairs 114, with each pair having a positive electrode and a negative electrode, and a power source 116, a processor 118, a Respiration Monitoring Device (“RMD”) 120, an optional Posture Indication Device 130, an optional Smart Controller 140 with a display 142 (e.g., a smartphone), and an acknowledgment button 144, and an optional Fob 150 with one or more buttons 152.

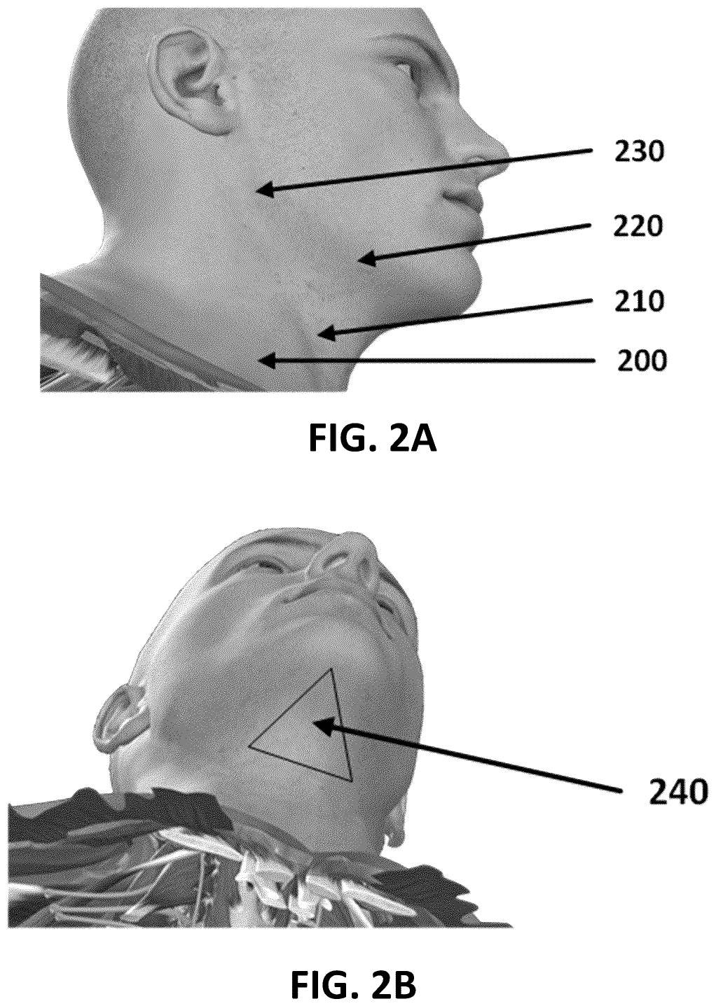

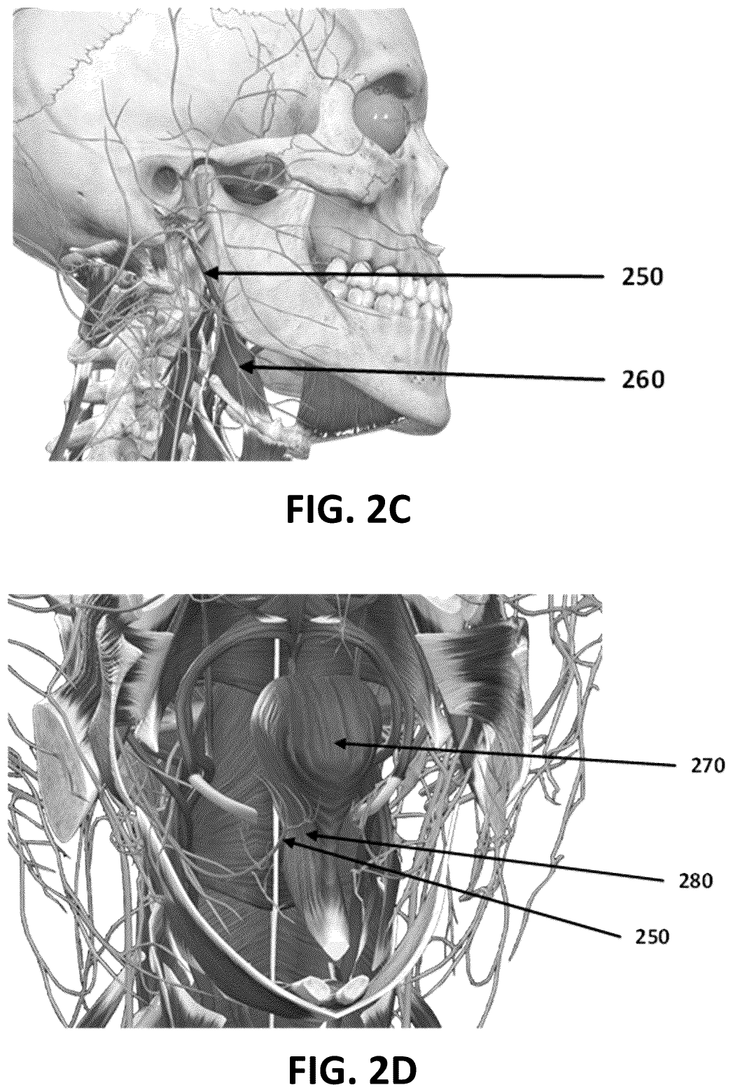

FIG. 2A shows a User 200, with a Neck 210, a Jaw Line 220, and an area behind the mandible 230.

FIG. 2B shows the User 200 and a submental triangle 240 below the chin.

FIG. 2C shows the User 200 and an internal view of the hypoglossal nerve 250 and the sublingual nerve 260.

FIG. 2D shows the User 200 and an internal view of the hypoglossal nerve 250, and the genioglossus muscle 280 under the tongue 270.

In FIG. 1, patch 110 is in a shape to conform to a selected dermis surface to be electronically effective at stimulating the hypoglossal nerve 250 and to monitoring breathing. Patch 110 can be used for apnea detection as well as for delivering electrical stimulation. Patch 110 is electronically most effective for stimulation when the positive and negative electrodes are placed axially along the path of the nerve in contrast to transversely across the path of the nerve, which is not as electronically effective.

The patch 110 shape is designed to minimize discomfort for the User 200 when affixed in the target location.

Patch 110 includes one or more sensors that measure internal features or biometrics of the User in the neck area. These measurements are used to orient and place the patch 110 most accurately in the target location and to monitor biometrics related to breathing. The sensor data is communicated to one or more of the Smart Controller 140, and the Fob 150; and is used by patch 110.

Additional sensor measurements are used to feedback biometric information in real time during the course of the sleep period in embodiments. This data is used by the Smart Controller 140 to confirm that a predicted but untreated apnea episode actually occurred, and/or to confirm that a predicted and treated apnea episode was cut short or eliminated by the treatment. This feedback data may be used to continuously calculate quality of sleep with a running AHI number and other metrics such as PPG (photoplethysmography) or Oxygen Desaturation, and may be used to adapt the treatment parameters to improve the success rate at eliminating apnea episodes.

The apnea treatment may be applied by other devices or systems outside the scope system 100, for example a CPAP machine.

In an example, patch 110 measures breathing and simultaneously measures blood oxygen level (SpO2). Breathing may be measured as an audio signal using one or more microphones. Breathing may be measured as a pressure signal using a cannula. Breathing may be measured as a temperature signal using a cannula.

In an example, the one or more microphones for breathing measurement are positioned on the throat and/or submental region. The audio is picked up through the tissue from the movement of air through the tracheal region on each inhale and exhale cycle, or breath.

In an example, the one or more microphones for breathing measurement are positioned on one or both sides of the nose. In an example, the one or more cannula for breathing measurement are positioned at the nares. In an example, the one or more cannula are positioned at the upper or lower lip. In some examples, an indication such as LED or vibration is sent to the User to assist them in placing patch 110.

Patch 110 in examples can be any type of device that can be fixedly attached to a user, using adhesive in some examples, and includes a processor/controller and instructions that are executed by the processor, or a hardware implementation without software instructions, as well as electrodes that apply an electrical stimulation to the surface of the user's skin, and associated electrical circuitry. Patch 110 in one example provides topical nerve or tissue activation/stimulation on the user to provide benefits to the user, including treatment for OSA.

Patch 110 in one example can include a flexible substrate, a malleable dermis conforming bottom surface of the substrate including adhesive and adapted to contact the dermis, a flexible top outer surface of the substrate approximately parallel to the bottom surface, a plurality of electrodes positioned on the patch proximal to the bottom surface and located beneath the top outer surface and directly contacting the flexible substrate, electronic circuitry (as disclosed herein) embedded in the patch and located beneath the top outer surface and integrated as a system on a chip that is directly contacting the flexible substrate, the electronic circuitry integrated as a system on a chip or discrete components and including an electrical signal generator integral to the malleable dermis conforming bottom surface configured to electrically activate the one or more electrodes, a signal activator coupled to the electrical signal generator, a nerve/tissue stimulation sensor that provides feedback in response to a stimulation of one or more nerves or tissues, an antenna configured to communicate with a remote activation device, a power source in electrical communication with the electrical signal generator, and the signal activator, where the signal activator is configured to activate in response to receipt of a communication with the activation device by the antenna and the electrical signal generator configured to generate one or more electrical stimuli in response to activation by the signal activator, and the electrical stimuli configured to stimulate one or more nerves or tissues of a user wearing patch 110 at least at one location proximate to patch 110. Additional details of examples of patch 110 beyond the novel details disclosed herein are disclosed in U.S. Pat. No. 10,016,600, entitled “Topical Neurological Stimulation”, the disclosure of which is hereby incorporated by reference.

FIGS. 2A and 2B show how the patch 110 is designed to be placed on either side of the neck, or on the jaw in one of several locations, such as the submental triangle 240, below the jawline 220, or behind the mandible 230; situated to accurately monitor biometrics related to breathing.

### Detection Using Respiration Monitoring Device

The function of Respiration Monitoring Device (“RMD”) 120 is to detect occurrences of interrupted breathing due to an apnea episode or hypopnea episode, and to notify one or more of patch 110, or the Smart Controller 140, or the Fob 150 of such an episode.

OSA has traditionally been diagnosed through a sleep study, or polysomnography, usually performed in a clinic setting with multiple electrodes monitoring multiple body parameters. Requests for home screening tests are rising because of comfort and cost issues. The STOPBANG scale has been used to provide guidance to those individuals assumed to be at high risk for moderate to severe OSA, with the acronym defined as Snoring; Tiredness in daytime; Observation by third party of stopped breathing; high blood Pressure; Body Mass Index (BMI) greater than 35; Age over 50; Neck circumference greater than 16 inches; and male Gender. A STOPBANG score of 3 or more indicates a home test or sleep study should be performed, especially since up to 80% of people with OSA do not know they have apnea.

The RMD 120 includes one or more of accelerometers and an audio sensing device. The accelerometer detects rhythmic movements of respiration, or the lack of such movement. The audio sensing device detects the sounds of air flowing through the throat, snoring, body motion in bed, and background noise.

The RMD 120 captures data from one or more of its accelerometers and audio sensing device as a time-based series of measurements, and sends the data to Smart Controller 140, which modifies the data series to derive the power signature of movement data from the accelerometers and the power signature of audio data from the audio sensors. By filtering the data to remove power components outside the frequencies which are indicative of inspiration and expiration, the Smart Controller deduces the series of breaths in the data series.

An example, the Smart Controller 140 measures the frequency content across multiple distinct frequency bands in a continuous manner, recording that stream of measurement data in its memory. The data is analyzed in real-time to detect patterns of frequency content that match two or more formants, similar to formants characteristic of human speech. The formants are chosen from the full set of speech formants to use those which indicate tongue position. By matching the real-time signature in the breathing audio to the signatures of the formants, the Smart Controller marks times when the tongue moves from a forward position to a rear position in the oral cavity. When the Smart Controller detects that the tongue has moved or is moving into the airway, the Smart Controller signals to patch 110 that an apnea episode has begun.

An example, the software converts the time domain audio signal into the frequency domain, such as by fast Fourier transform or digital Fourier transform or similar means, and creates a data sequence to show, at each sampled time step, the energy content of the audio at each specified frequency band.

An example, the frequency bands are determined at the beginning of the device usage from a default set.

An example, the frequency bands are determined at the beginning of the device usage during a training period, during which the device monitors the user and calculates the formant content for that specific user, followed by the diagnostic period, in which the audio data is distilled into formant energies according to the previously-determined formant definitions.

Following the previous example, the frequency bands continue to be adjusted during the diagnostic period, thereby using a closed-loop system to improve the distillation of apnea-related energies based on the real-time changes in the user's breathing, such as when the user changes sleeping positions, which may be detected by motion sensors on the device, or when the user's nasal or oral passageway changes as in congestion.

In some examples, the formant recognition is performed by firmware or software in the Neck TNA Device 110.

In some examples, the formant recognition is performed by firmware or software in the RMD 120.

In some examples the duration of each inspiration is measured by the RMD 120 to estimate the magnitude of the indrawn breath, the Obstructive Sleep Apnea Detection and Stimulation System 100 using this measurement to distinguish apnea events from hypopnea events. Thus, shorter, incomplete breaths will have a duration distinguishable from longer, complete breaths, using a threshold established by the Obstructive Sleep Apnea Detection and Stimulation System 100 or set and adjusted periodically by the Smart Controller 140, based on filtering of data to discern a minimum indrawn breath indication for the specific user.

The RMD 120 can be positioned at various locations on the body, depending upon which sensor and which body parameters are measured. For example, an RMD containing an accelerometer is positioned on the chest to detect rhythmic breathing patterns or on the neck in the submental region, or on the lower neck at the suprasternal notch; whereas an RMD containing an audio sensor can be positioned near the outside of the nasal passageway or on the neck in the submental region, or on the lower neck at the suprasternal notch.

In some examples, the RMD 120 is a separate unit from patch 110, is positioned at various anatomical locations around the body away from patch 110.

In some examples the RMD 120 is integrated within patch 110, and monitors specific body signals at the same location as patch 110.

### Example Obstructive Sleep Apnea Detection and Stimulation System

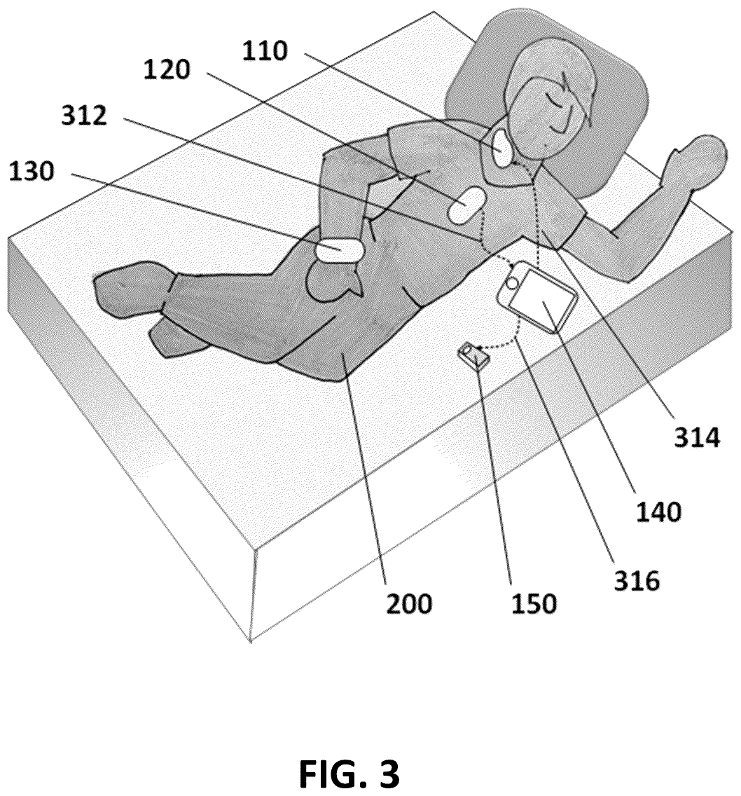

FIG. 3 illustrates an Obstructive Sleep Apnea Detection and Stimulation System that includes a patch 110, and a Respiration Monitoring Device (“RMD”) 120. The RMD may be a separate unit or integrated within patch 110. It may also include a Smart Controller 140 or Fob 150. The User 200 may indicate to patch 110 or Smart Controller 140 or the Fob 150 directly when the User is beginning a Sleep Period and again when the User is ending a Sleep Period. During the Sleep Period, when the RMD senses an OSA episode, the RMD then signals the Smart Controller or Fob that the apnea be recorded and optionally suppressed using patch 110, then the Smart Controller signals to patch 110 to activate the nerve, then the Smart Controller optionally signals to the Fob to record such an activation event that the apnea is suppressed for a period of time.

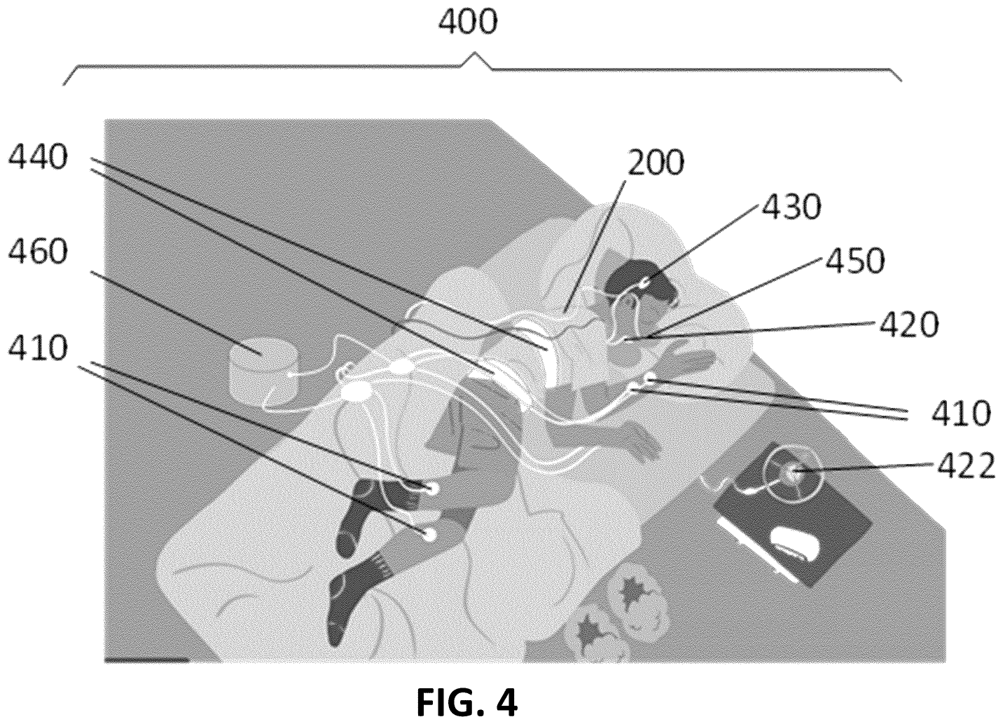

FIG. 4 illustrates a conventional polysomnography (“PSG”) system 400 that may include a number of sensors and wires attached to the User 200 while sleeping, including a set of Leg Movement Sensors 410 with wires, a Snoring Sensor Microphone 420, an Ambient Noise Microphone 422, an EEG Electrode 430, one or more Breathing Detection Belts 440, and an Airway Sensor 450. As may be seen in the figure, the wires connecting the sensors to the PSG Controller 460 may interfere with the User's sleep, or the measurements may be adversely affected by User movement, or the measurements may be stopped due to disconnection of one or more sensor due to User movement.

### Configurations

As disclosed, a device such as patch 110, applied to the skin on the face, head or neck, may be used to detect sleep apnea episodes and, with electrodes in the same or similar device design, may be used to treat the sleep apnea episodes such that the episodes are reduced and the effect of sleep apnea on sleep and other health aspects of the user is reduced. However, due to the variability in human physiology from one user to another, such as the shapes of facial features or the underlying location of nerves related to the treatment of sleep apnea episodes, the effectiveness of a such device or devices is limited. When the device is not placed in the optimum location, its ability to detect and/or treat the apnea is limited. The user may experience limited benefit. Therefore, embodiments automatically select a subset of pairs of electrodes from a possible group of pairs of electrodes and adjust stimulation parameters to treat an OSA episode.

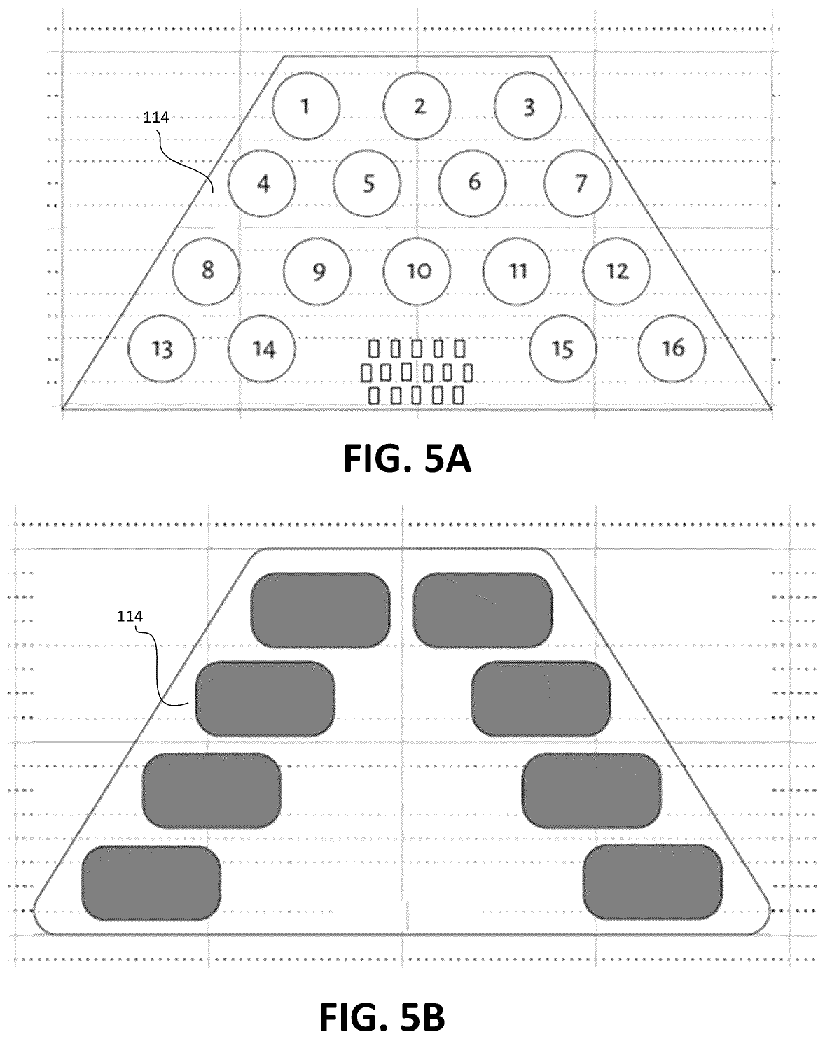

FIGS. 5A and 5B illustrate numbered electrodes 114 arranged in a rectangular matrix as an example of a multi-electrode array on patch 110 in accordance to embodiments.

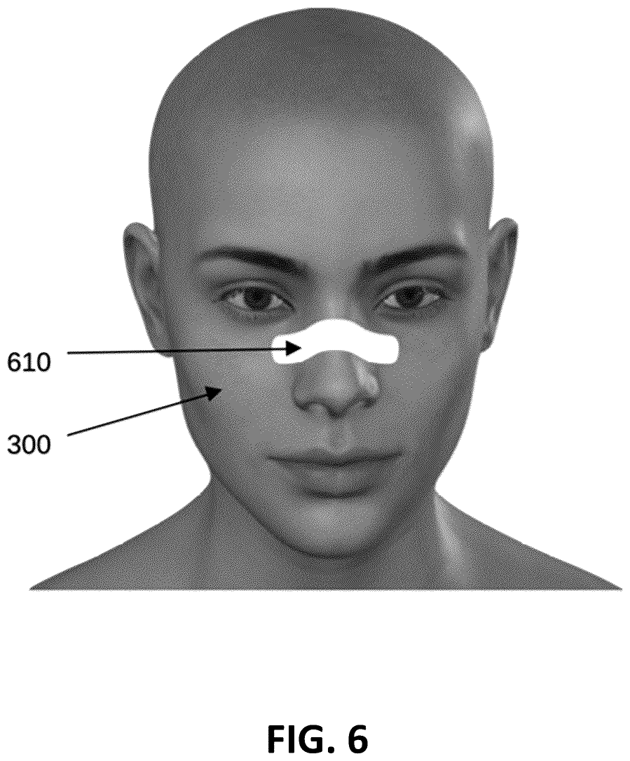

FIG. 6 shows a Nasal Device 610 as an example shape and placement for patch 110 in accordance to embodiments. In FIG. 6, Nasal Device 610 is shown affixed to the bridge of the nose of a user 300, with sensors on each of the left and right side of the nose.

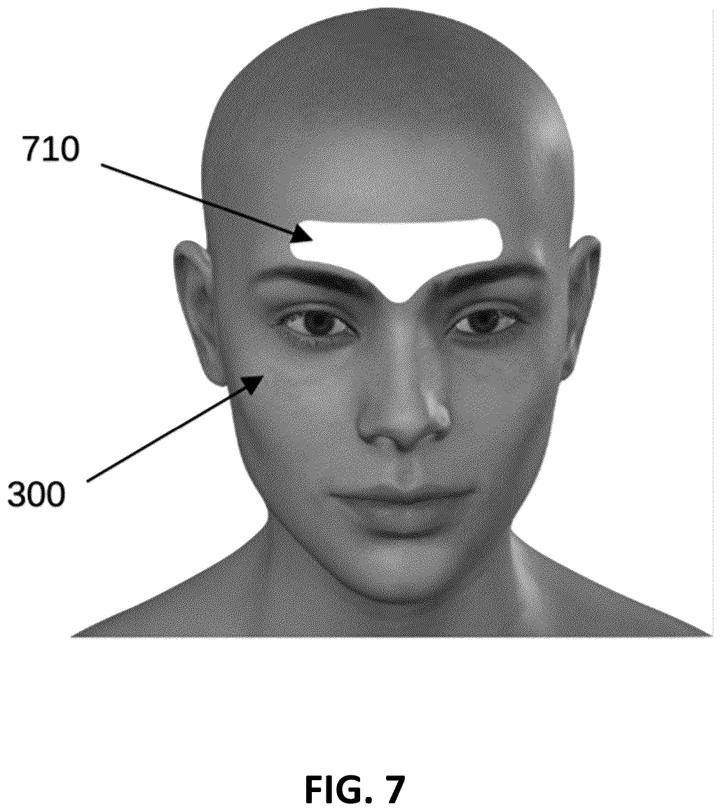

FIG. 7 shows a Forehead Device 710 as an example shape and placement for patch 110 in accordance to embodiments. Forehead Device 710 is shown affixed to a forehead of a user 300, with sensors on the forehead and optionally extending down the ridge of the nose.

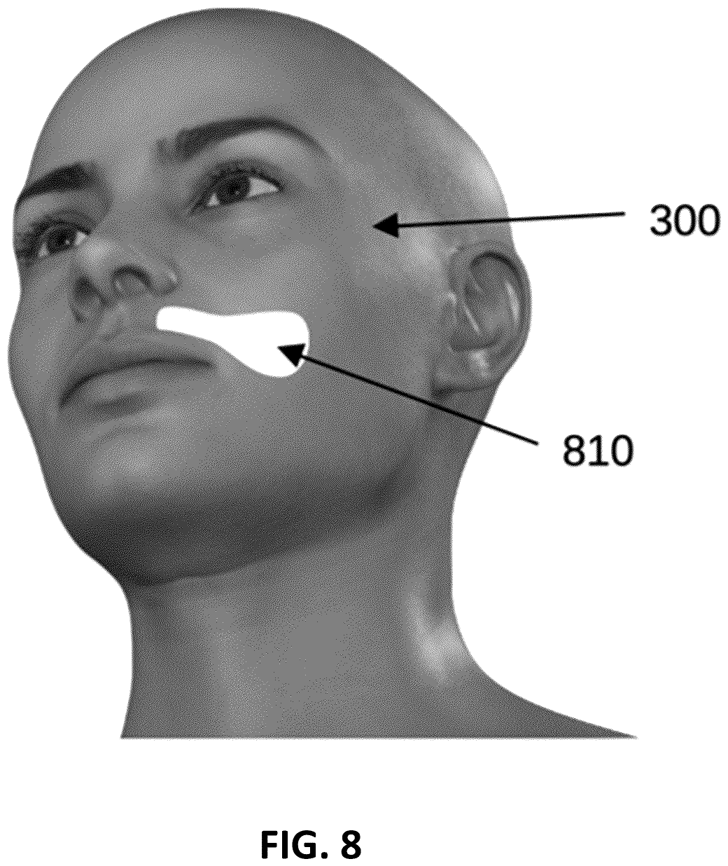

FIG. 8 shows a Facial Device 810 as an example shape and placement for patch 110 in accordance to embodiments. Facial Device 810 is shown affixed to a right cheek of a user 300, with sensors on the cheek and optionally extending down to the submental triangle.

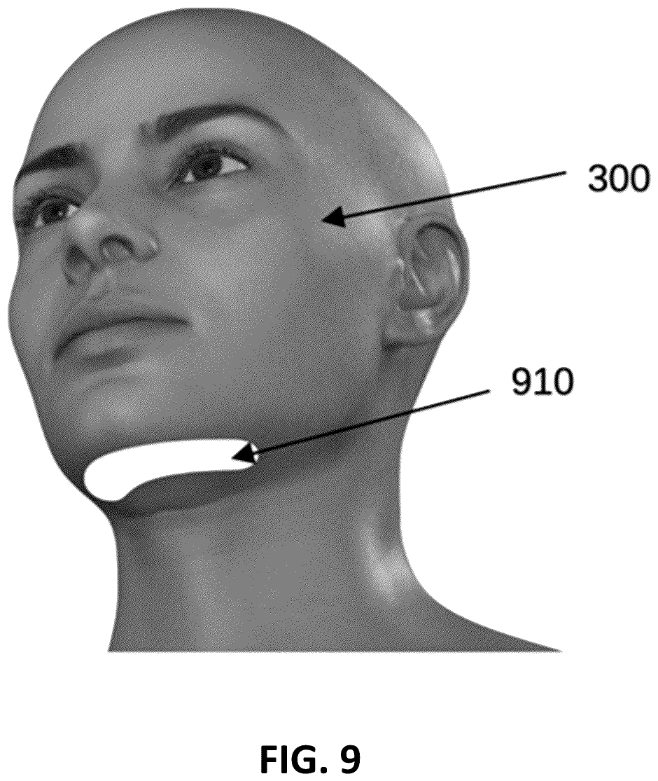

FIG. 9 shows a Submental Device 910 as an example shape and placement for patch 110 in accordance to embodiments. Submental Device 910 is shown affixed to a submental triangle of a user 300.

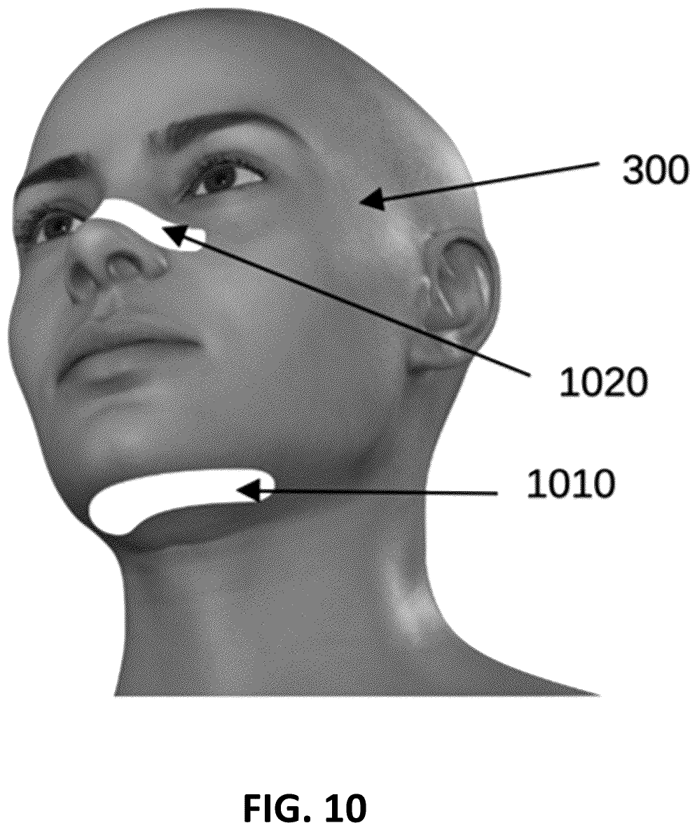

FIG. 10 shows a system with both Nasal Device 1020 and Submental Device 1010 as example shapes and placements for patch 110 in accordance to embodiments. Nasal Device 1020 and Submental Device 1010 are combined into a Two-Device System with each of the Nasal and Submental Devices communicating with each other and with Smart Controller 140 to monitor user 300 for apnea episodes and apply stimulation.

### Electrode Stimulation

As disclosed, with embodiments, after detection, electrical stimulation/stimuli may be required at one or more nerves to open the airway during an apnea episode. A set of muscles controls the various parts of the upper airway and each is controlled by a nerve or nerves. The hyoid bone is moved forward by contraction of the digastric and the geniohyoid muscles and elevated by contraction of the mylohyoid muscle. The tongue has intrinsic and extrinsic muscles, all of which affect the opening of the airway.

Various pairs of electrodes may directly or indirectly preserve or gain patency of the upper airway with embodiments.

In an example, as shown in FIG. 5, electrodes #3 and #16 may be used to ipsilaterally stimulate the left geniohyoid muscle.

In an example, electrodes #4 and #7 acting as anodes with electrodes #9 and #11 acting as cathodes may stimulate the mylohyoid muscle and/or the inferior alveolar nerve via the mandibular nerve, which will flatten the floor of the oral cavity and elevate the hyoid bone and depress the mandible and separate the teeth in the oral cavity.

In an example, patch 110 sequentially stimulates with each of its set of Electrode Pairs or combinations of Electrodes 114 while Smart Controller 140 indicates the stimulation to the user through the user interface. The user notes the involuntary response of the tongue, or lack of response, to Smart Controller 140. Smart Controller 140 takes note of the Electrode Pairs which are most effective in creating an involuntary response of the tongue. This response may be created by different Electrode Pairs according to the exact placement of patch 110 on the user or on variations in physiology from user to user.

In an example, two or more Electrode Pairs 114 are found to be most effective, these pairs affecting one or more nerve. In some examples, the stimulation timing, amplitude and duration may be different for each Electrode Pair found to be effective. In embodiments, the electrodes in the form of adhesive conductive pads may be applied to the skin, and electrical stimuli is passed from the adhesive pads into the tissues. The stimuli may typically be trains of voltage-regulated square waves at frequencies between 15 and 50 Hz with currents between 0.01 and 100 mA. In other examples, the stimuli includes square waves having an amplitude between 10 and 100 volts, pulse widths between 100 and 500 microseconds, and a pulse repetition rate of between 2 and 60 pulses per second.

In an example, the efficacious Electrode Pair or Pairs may require the use of higher amplitude or longer duration stimulation to be optimally effective in opening the airway. These amplitude and/or duration settings may be found algorithmically by patch 110, with or without communication with Smart Controller 140.

A “pair” of electrodes can include an evenly matched pair of anodes and cathodes, or an uneven non-symmetrical arrangement of anodes/cathodes. For example, in one embodiment, an electrode could be in any of three states: anode/cathode/not connected where the stimulating signal is bi-phasic, causing the anode/cathode relationship to flip for every pulse given. Therefore, some configurations of the electrode array that includes “pairs” of electrodes may include, for example, 5 anodes and 3 cathodes, or 2 anodes and 6 cathodes, where there is not an even matched number of anodes/cathodes.

In an example, patch 110 sequentially stimulates with each of its set of Electrode Pairs 114 while patch 110's one or more sensors measure the physiological reaction to each stimulation, noting those measurements which indicate a response in the one or more muscles which affect the airway opening. This sequential stimulation may also include adjustment of timing, amplitude, duration and similar parameters applied to each electrical stimulation, all in a process of determining the most effective stimulation configuration. This process may be performed while the user is asleep, thereby measuring effectiveness without user participation and during the actual sleep experience. In some examples, the process may be performed at the beginning of the sleep process to adjust stimulation for patch placement, user sleep position, etc. In some examples, the process may be repeated through the period of sleep, such as in real time or at intervals such as once per hour, to account for changes in effectiveness as the user transitions between sleep phases or changes their sleep position.

In an example, the stimulations may reduce the drop in blood oxygen level (i.e., SpO2) since normal breathing is restored and an apnea event is avoided or curtailed, thereby providing a measurement in the SpO2 to indicate in a closed-loop manner the effectiveness of one or more particular electrode selection and one or more application of electrical stimulation.

Stimulation may be used to cut short an apnea episode. Stimulation is triggered by a signal sent from patch 110 when patch 110 predicts an apnea episode.

Similarly, other types of apnea prevention equipment may be triggered to intervene using a signal from patch 110. For example, CPAP and APAP systems may adjust their settings based on triggering from patch 110.

As disclosed, embodiments include a multi-electrode array 114 whose dynamically reconfigurable properties will allow for the personalization of stimulation treatments to accommodate varied patient physiologies (e.g., varied BMI, adipose tissues, neuropathy conditions, etc.), health conditions (e.g., COPD, dysphagia, etc.), and reactions to treatments (e.g., arousal threshold), in view of the desire keep the treatment stimulations below an arousal threshold (i.e., sub-arousal, skin irritation). In embodiments, multi-electrode array 114 an array of electrodes coupled to the substrate of patch 110, where the array of electrodes include a plurality of electrode pairs in different geometric positions on the substrate

Additional characteristics that may be considered when automatically selecting one or more pairs of electrodes from a multi-electrode array include (1) whether the treatment is in the submental area; (2) whether the treatment is specifically for apnea; (3) whether the treatment can be adaptive during the treatment (e.g., breaking the treatment up into 20 minute segments, each with its own regimen) or between treatments (e.g., adjusting any analytical models or retraining machine learning models between treatments); (4) responsive to a feedback loop to determine the appropriate treatment per patient (e.g., monitoring biometrics of the patient such as SpO2, heart rate, body temperature, breathing characteristics to establish the feedback loop and to determine an appropriate treatment regimen, etc. With embodiments, the multi-electrode array allows for accommodating misplaced patch locations applied by the user.

Embodiments solve the technical problem of limited effectiveness of a patch, placed on the user's skin, to detect and/or treat apnea, when the patch placement is not optimal. A matrix of electrodes is used, in which different pairs or sets of electrodes are activated by the on-patch controller, and/or the waveform is adjusted, using feedback from the awake user or using measurements fed back to the controller, to determine which electrode selection and/or which waveform configuration is best for detecting and/or suppressing apnea events.

### Data Collection

In some examples, Smart Controller 140 records the measurements made and the stimulations performed during the adjustment process. The measurements will includes one or more of: breathing parameters such as oxygen desaturation or respiration rate; electrical signals such as EMG, EEG, or ECG; external signals from other measurement devices; or user or healthcare provider inputs. In some examples, Smart Controller 140 sends the accumulated data to a permanent storage system such as cloud storage. In some examples, this accumulated data is used across one user's sleep experiences to adjust the treatment protocol. In some examples, this accumulated data is combined with data from other users across a population of users to adjust the treatment protocol such as adjusting a machine learning model, retraining an artificial intelligence model, noting statistics for infrequently used configurations which may affect future patch 110 designs.

In some examples, analysis of measurements from one or both of the Smart Controller 140 and patch 110 may be performed by processing in a remote server, in the cloud, or on a computer separate from Smart Controller 140 but local to the ser, such as a personal computer.

In some examples, Apnea Detection and Stimulation System 100 measures the user's sleep schedule over a period of days or weeks or longer, noting the clock time when the user begins the sleep period and the clock time when the User wakes during or at the end of the sleep period. System 100 analyzes this data and determines the most effective clock times to activate the Apnea Detection and Stimulation System 100.

### Prediction

In embodiments, system 100 may be used in one or more of the following modes. System 100 may analyze real time data to predict or anticipate an apnea event. Embodiments use artificial intelligence (“AI”) and machine learning (“ML”), based on training from sleep data, to calculate the likelihood of an apnea episode onset continuously during the sleep period. The percent of false positives is minimized while the percent of false negatives is also minimized. Each predicted episode is logged in the collected data. Embodiments may, after predicting an episode, signal to the electrical stimulation system to stimulate the user with the intention of opening the airway and continuing normal breathing with a short or eliminated cessation period. The stimulation may or may not be effective, most often depending on the specific physiology of the user. Embodiments may, after electrical stimulation, measure one or more biometric parameters and use that data to determine whether the breathing interruption was stopped, cut short or allowed to proceed. Embodiments may further use the statistics of success or failure from this subject or a cohort of large numbers of subjects to adjust the treatment protocol for subsequent electrical stimulations.

An apnea episode begins when the user stops breathing. The regular cadence of breaths is interrupted. After a delay subject to the user's physiology and sleep environment, the blood oxygen level begins to drop and a variety of deleterious effects begin to affect the user. Longer apnea episodes have larger effects. In the case of a hypopnea event, breathing changes to very shallow breaths which are not enough to sustain complete oxygen saturation. The harmful effects occur during hypopnea as well as during apnea. A series of apnea events or hypopnea events, such as every two minutes (i.e., an AHI of 30), cause cumulative harm to the user as blood oxygenation and stress on the cardiovascular system is not completely relieved between events. For these reasons, eliminating an apnea episode (or hypopnea episode), or cutting its duration short, will reduce the harm to the user.

Embodiments collect audio data in real time and analyze it through one or more algorithmic processing step to determine if airflow has stopped, indicating a missed breath. A machine learning model that forms part of system 100 may be used to determine if the audio event (i.e., a “flatline” event) is a predictor of apnea or hypopnea. The machine learning model makes this determination having been trained on datasets of sleep data. In embodiments, the machine learning model improves its confidence in predicting events by incorporating the feedback data from other, post-event biometrics.

### Closed-Loop Confirmation

Embodiments measure one or more parameters from the user and feed that real time data back to the prediction algorithms and machine learning model. For example, patch 110 measures blood oxygen saturation (SpO2) level at the user, such as in the fingertip, earlobe, nares or forehead. Since the blood oxygen level drops slowly after breathing is interrupted, the low point of SpO2 occurs many seconds after the start of the apnea event and may occur many seconds after the end of the apnea event.

If system 100 is used to monitor sleep without apnea treatment, then the SpO2 measurements indicate that an apnea event has or has not occurred. If the SpO2 indicates an apnea event, and the system 100 predicted the event, then that is a true positive prediction. If the SpO2 indicates no apnea event, and system 100 predicted an event, then that is a false positive. If the SpO2 indicates an apnea event, and system 100 did not predict the event, then that is a false negative. If the SpO2 did not indicate an apnea event, and system 100 did not predict the event, then that is a true negative. Embodiments use the feedback data to reduce the number of false positives and false negatives, while maintaining accurate true positives and true negatives.

If system 100 is used to monitor sleep and apply apnea treatment, then the SpO2 measurements indicate that an apnea event which occurred has or has not been suppressed. If the SpO2 level drops after system 100 predicts and treats for an event, then system 100 failed to suppress the event. A “drop” is a reduction in blood oxygen level, measured and expressed in terms of percentage of normally fully oxygenated blood. System 100 may define a drop as a specific number in percentage terms, such as a 3% or 4% drop. The treatment configuration may need to be adjusted such as, for example, higher amplitude electrical pulses. If the SpO2 level does not drop after system 100 predicts and treats for an event, then system 100 succeeded in suppressing the event. The success and failure rate of system 100 are assessed in real time and the protocol is adjusted to improve success rate and to reduce failures. In embodiments, other parametrics may be used to feed back success and failure information after apnea treatments and to improve false positive and false negative performance in prediction.

In embodiments, the timestamps for data in the feedback loop are matched to timestamps in the prediction and treatment protocols. When the feedback data is delayed from the treatment time, such as with SpO2, then the timestamps for the SpO2 data may be changed to match those of the treatment.

In some examples, the feedback data originates in sensors in patch 100 affixed to the user. In some examples, the feedback data originates in sensors in Smart Controller 140. In some examples, the feedback data originates in sensors in secondary devices at or near the user during sleep, such as a fingertip pulse oximeter.

As disclosed, embodiments, in response to treatment feedback, adjust stimulation treatment parameters (e.g., frequency, amplitude, pulse duration, etc.) of the stimulation, the location of the stimulation (e.g., which electrode configuration is chosen), or the sequence of future stimulations. The feedback of biometrics to adjust the stimulation treatment in real-time is disclosed above. One or more nerves or tissues can be stimulated, not just a single nerve as the case with many other stimulation systems. As an example, SpO2 in embodiments is a measured parameter that is used to explain how the treatment will adapt using the measured SpO2 level following a stimulation to determine efficacy or non-efficacy of the treatment.

Embodiments use machine learning and artificial intelligence to supplement the algorithmic treatment of biometric measurements, such as breathing audio, to improve detection of apnea events. Embodiments reduce the number of false positives and false negatives, thereby optimizing both the assessment of apnea severity (“AHI”) and the treatment of events without excessive or inconsistent use of stimulation.

### Cloud-Based or Remote Apnea Detection and Treatment System

Embodiments may be used to predict an OSA episode in real time and then send a signal in real-time to sleep apnea prevention systems to prevent the episode from proceeding, thereby limiting it to less than ten seconds such that it does not count in the calculation of AHI. Shortened or eliminated apnea episodes reduce the load on the user's physiology. The prediction of each apnea episode is confirmed by the system using biometric measurements in parallel with the prediction and treatment process such that no sleep expert annotations are needed as the correlation can confirm apnea episodes as well as confirming the elimination of apnea episodes, all in real time.

System 100 monitors biometrics related to breathing, and optionally, a computing resource able to analyze the breathing data and send improved parametric values to the device on the user's skin to improve the detection capabilities for that specific user, in a closed-loop system. Embodiments may also include a computing resource to send signals to a device or devices to prevent the apnea episode from progressing. The system may include one or more sensors to monitor the effectiveness of the electrical stimulation and, through a computing resource, adapt the parameters used in prediction to improve the success rate of prediction. This closed loop adaptation of prediction through the use of adjustable parameters improves upon open loop systems.

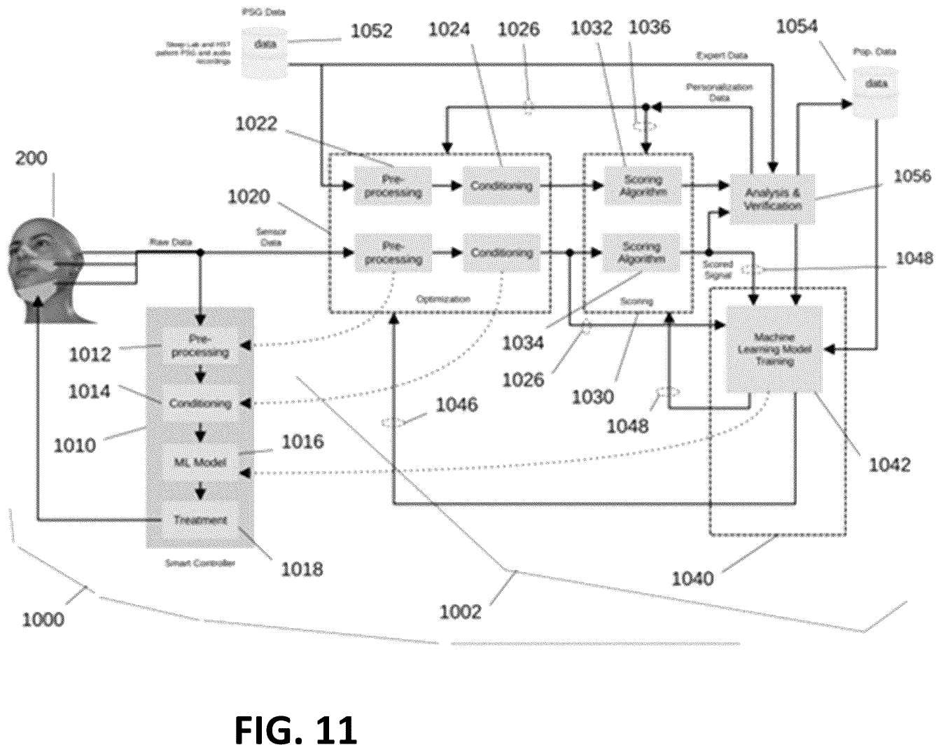

FIG. 11 illustrates a Apnea Detection and Stimulation System (ADSS) 1000, with a Data Handling System 1002, a Smart Controller 1010, a Data Optimization Stage 1020, a Data Scoring Stage 1030, a Machine Model Stage 1040, an Analysis and Verification Stage 1050, and a User 200 in accordance to embodiments. Data Handling System 1002 may be cloud based, on-premise, or otherwise remote from patch 110 and smart controller 140.

in FIG. 11, the User 300 wears one or patch 110 on their person during a night of sleep, as shown by the examples in FIG. 6-10. Data in its raw form, Raw Data 1019, is collected by both the Smart Controller 1010 and the Data Optimization Stage 1020. Data passes through similar portions of the optimization stage in both the Smart Controller and the Data Handling System. Pre-processing and Conditioning modify the data, such as to remove noise, improve quality of the signal, and similar signal processing steps. The output Sensor Data passes to the Data Scoring Stage 1030, and to the Machine Model Stage 1040.

In an example, the input data to the Data Optimization Stage 1020 comes from an external PSG Data database 1052. The data processing is performed on data collected at some earlier time. Data optimization may be divided into algorithmic processing in a Cloud Data Pre-Processing Block 1022 and a Cloud Data Conditioning Block 1024.

In an example, the input data to the Data Optimization Stage 1020 comes from the one or more patches 110 on User 200. The data processing is performed in real time.

The Data Scoring Stage 1030 examines the stream of input data and makes a prediction for each apnea event it determines exceeds a likeliness threshold in its algorithmic analysis.

In embodiments, the Machine Learning Stage 1040 utilizes three type of input data streams to train a Machine Learning Model 1042. The Machine Learning Model 1042 uses the machine learning model to infer when the next apnea event will occur. One of the three types of input data streams is Conditioned Sensor Data 1026 as output from the Data Optimization Stage 1020. A second type of input data is Scored Event Data 1036 as output from the Data Scoring Stage 1030. A third type of input data is Event Annotation Data 1056 from the Analysis and Verification Stage 1050.

In an example, the Machine Learning Stage 1040 trains its Machine Learning Model 1042 using a combination of Conditioned Sensor Data 1026 and Event Annotation Data 1056. Using one or more neural networks, the Machine Learning Stage constructs the Machine Learning Model by optimizing the correlation between the series of events annotated by an outside evaluation system. For example, the expert annotation is data collected from human sleep experts performed during a sleep study on the combined data from the user's sleep period.

In an example, the Machine Learning Stage 1040 trains its Machine Learning Model 1042 using a combination of Conditioned Sensor Data 1026 and Scored Event Data 1036.

In an example, the expert annotation is data extracted from correlation to an accurate, but lagging indicator of an apnea event. This type of real-time but lagging data cannot be used to predict an apnea event because the change in the data does not begin until the apnea event is well underway or has passed. One example is blood oxygen level, SpO2, which falls when breathing stops, yet is not measured as a drop until some seconds after the first apnea event. A second example is heart rate variation, which is affected directly by stopped breathing, yet not for some seconds after the first apnea event.

It will be apparent, however, to one skilled in the art that there are other measurements which lag the actual apnea event and are not useful for predicting such events yet are useful in qualifying after the inception of an apnea event whether a predicted event actually occurred.

An ML-to-Sensor Feedback 1046 is a feedback path which improves the Data Optimization Stage 1020 one or more algorithms iteratively. The optimization and/or scoring algorithms are modified to optimize the statistical correlation between scored and expert annotated data.

An ML-to-Sensor Feedback 1048 is a feedback path which improves the Data Scoring Stage 1030 one or more algorithms iteratively. The optimization and/or scoring algorithms are modified to optimize the statistical correlation between scored and expert annotated data or between scored and a lagging indicator data of an apnea event.

The Data Optimization Stage 1020 and the Data Scoring Stage 1030 use a finite set of modifiable parameters to change the Raw Data 1019 into data which can be judged to predict apnea events.

In an example, the Raw Data 1019 is audio data from breathing and the modifiable parameters are allowed background noise level and background interruption amplitude, where background noise level sets a value under which the audio signal is considered to be noise and not a breath, and background interruption amplitude sets a level above which a loud noise is considered unrelated to breathing.

In an example, the Raw Data 1019 is audio data from breathing and the modifiable parameters are variance and length, where variance is a measure of the variability of the audio signal on the timeline, and length is a selection of the number of consecutive seconds of signal seen as null to be judged as a lack of breath. An apnea event has ten or more seconds of length. To predict an apnea event, the Data Scoring Stage 1030 is set to watch for less than ten seconds of quiet signal. One example of variance may be the amplitude of the signal in the time domain wherein amplitude below a threshold is judged as quiet. A second example of variance may be energy of the signal in the frequency domain wherein energy from second to second below a minimum energy level across a specified range of audio frequencies is judged to be quiet.

Each of the set of modifiable parameters may be run through a series of values or settings in a range which is meaningful. In the aforementioned example, the amplitude range is the range of signal amplitudes seen from breaths in the seconds, minutes, hours or days preceding the judging of quietness in the present breath's timeframe. In the aforementioned example, the energy level is the range of energies in the frequency domain, with a finite range of frequency. Since each modifiable parameter is changed through a finite range in discrete steps, it follows that the permutations and combinations of these parameters forms a finite set of possible settings.

The Analysis and Verification Stage 1050 permutes the modifiable parameters through their ranges, sending a Set of Settings 1029 back into the Data Scoring Stage 1030 and then assessing the quality of the prediction.

In an example, the quality of the prediction is a combination of number of true positives, number of false positives, number of true negatives, and number of false negatives, where true positives are predicted apnea events at times when there is an actual apnea event, false positives are predicted apnea events at times when there is not an actual apnea event, true negatives are negative predictions of apnea events at times when there is no actual apnea event, and false negatives are negative predictions of apnea events at times when there is an actual apnea event.

The usefulness of embodiments is to predict accurately with a high true positive and a high true negative score while also having a low false positive and a low false negative score.

In an example, the usefulness embodiments is shown in an AHI score for the patient which meets or exceeds the AHI score of human sleep experts' scores.

In an example, a high true positive score and a low false positive score results in treatment of predicted apnea events only when an apnea event actually would have occurred if allowed to proceed.

In an example, a high true negative score and a low false negative score results in lack of treatment only when an apnea event would not have occurred.

The Analysis and Verification Stage 1056 may assess the quality of the predictions by means of true and false positives and true and false negatives, or other means, and thereby determine the optimum Set of Settings for the modifiable parameters. This optimum Set of Settings may be different from user to user, and/or from night to night for a specific user, and/or from hour to hour in one night for a specific user, and/or from one sleep stage to another sleep stage in one night for a particular user.

It will be apparent, however, to one skilled in the art that there are other scenarios in which variations in the sleep situation for the user will affect the choice of optimum set of settings for the modifiable parameters.

The Analysis and Verification Stage 1056 may feedback the optimum Set of Settings into the Data Optimization Stage 1020 and/or the Data Scoring Stage 1030. This feedback path may be used to improve the performance of one or both of these stages.

Through the use of these feedback paths, the algorithmic stages of data handling are improved. With improvements in the data conditioning, the machine learning stage improves its precision and recall, thereby marking all of the real apnea events passing through in real time and no extra nor missed apnea events.

In an example, the machine learning model improves its confidence in predicting events by incorporating the feedback data from other, post-event biometrics.

Smart Controller 140 has one or more of a Pre-Processing Block 1012, a Conditioning Block 1014, an ML Model Block 1016 and a Treatment Block 1018.

In an example, the Pre-Processing Block 1012 is a copy or a modified copy of the Pre-Processing Block 1022.

In an example, the Conditioning Block 1014 is a copy or a modified copy of the Conditioning Block 1032.

In an example, the configuration parameters for the conditioning of pre-processed data are copied from the Cloud Data Conditioning Block 1022 to the Device Data Conditioning Block 1012.

In an example, the ML Model Block 1016 is a copy or a modified copy of the Machine Learning Model 1042.

In an example, the Machine Learning Mode 1042 is transcribed into the ML Model Block 1016 as software and/or firmware in the Smart Controller 1010.

In an example, the configuration parameters, termed “weights” in some models, are copied from the Machine Learning Model 1042 to the ML Model Block 1016. Copying the parameters may be faster and no less effective than copying the entire model.

As shown in FIG. 11, embodiments use the output of the analytical algorithms to train (and re-train based on feedback) an ML model. The results of the predictive ML model are used to establish a treatment regimen for a patient and the post stimulation measured biometric data to improve the training of the ML model. Embodiments use of the post-stimulation measured biometric data to simultaneously optimize the performance of the analytical algorithms and the ML model to achieve more overall accuracy as evidenced by ML precision and recall metrics.

The data analysis model as shown in FIG. 11 in embodiments includes (1) signal preprocessing; (2) data normalization; (3) analytical analysis of the breathing signal for detection of apnea events; (4) ML training; and (5) the use of ML for apnea treatment. Embodiments also reinforce the closed-loop feedback using biometric parameters (e.g., SpO2) to optimize the apnea treatment.

Sleep Apnea Adaptive Detection and Treatment System with Coincident Pharmaceutical Treatment

In some known solutions, OSA can be treated with pharmaceuticals. Such drug treatments fall into three categories: promoting wakefulness, addressing co-existing conditions (such as obesity), and aiding in sleep. Each of these drug approaches has limitations in terms of side effects or limited effectiveness at eliminating OSA events.

Embodiments of the invention are able to operate coincidentally with a pharmaceutical treatment, enhancing the effectiveness of the one or more medications. With the enhanced effectiveness, the dosage of the one or more drugs may be modified, thereby reducing drug side effects. Embodiments are able to reduce the need for pharmaceutical treatment by eliminating apnea events through electrical stimulation. Dosages may be tailored to the lowered apnea event frequency and/or intensity, thereby reducing drug side effects.

In some cases, the electrical stimulation provided embodiments may sufficiently lower the frequency or intensity of apnea events that the pharmaceutical treatment may be stopped for one or more drugs, thereby eliminating the side effects of those drugs.

Embodiments are able to monitor sleep and apnea incidents on a night-by-night basis, continuously, thereby enabling the study of drug effectiveness across many nights of sleep rather than basing prescription of drugs on one night sleep studies.

The use of one or more drug may improve the effectiveness of electrical stimulation as applied by embodiments, thereby reducing the number of stimulations needed to maintain a low apnea incidence count. Such reduction in stimulations extends the battery life of the applied system. Such reduction may reduce the side effects of electrical stimulation.

Medications have been prescribed to improve the performance of the upper airway (UAW) during sleep. The medications include “Diamox”, “Strattera”, “Sunosi”, etc. Medications have also been prescribed to alleviate nasal congestion and are also used to reduced sleep apnea, including “Afrin”, “Neo-Synephrine”, “Sudafed”, etc. However, all of these medications are affiliated with side effects. The side effects may affect the user during the sleep period and/or during the wake period. Compliance with medication is improved by reducing side effects.

Embodiments that use patch 110 eliminate or reduce a portion of the apnea events during the sleep period, allowing for the use of modified drug levels. Embodiments eliminate a portion of the apnea events during each sleep period, allowing for the use of reduced drug levels. In some examples, medication levels may be reduced.

In some examples, a different medication regimen may be prescribed due to the lower number of apnea events. In some examples, medication may be eliminated. Some medications act directly on the central noradrenergic system in the brain, for example norepinephrine. The noradrenergic system has been related to stress-related symptoms, including post-traumatic stress disorder.

Embodiments use electrical stimulation to reduce the incidence of apnea events, thereby reducing the need for decongestants as an apnea treatment. With the reduced dosages of these medications, the side effects such as enlargement of the soft palate are reduced. As congestion in the airway, including the effect of the soft palate, aggravates sleep apnea, a reduction in swelling of the soft palate and other tissues reduces the incidence of apnea events.

In some examples, embodiments improve oral breathing by opening the airway even when the nasal passage is blocked by swelling. By reducing the incidence of apnea events and improving oral breathing, embodiments reduce the need for medications for nasal congestion, as the user is able to sleep with oral breathing. With reduced application of nasal congestion medication the side effects of such medication are reduced.

Further, compliance with medication is a major problem with drugs. Taking medications outside of the prescribed regimen will affect the desired performance of the prescribed medications. Lack of compliance generally means that a patient/user has not taken their medication, either accidentally because they forgot, or purposely because they are trying to self-titrate (i.e., control their dosages themselves). Embodiments can detect and adapt to changing apnea conditions that may be the result of compliance issues. This is “complementary medicine, CM”, the combination of drugs and electrical stimulation. By continually adapting its treatment regimen, embodiments can compensate for poor or erratic compliance.

Compliance with medication is improved by reducing side effects. In some examples, embodiments acts to monitor its own usage, such that occasions when the user does not use the stimulation function, for example by forgetting to apply a stimulation patch, that incident is recorded in the system. A report of performance against the planned use of detection and/or stimulation may be sent to the user, a medical advocate, a doctor, and similar persons.

In some examples, embodiments may be used to record and then report on incidents when the user fails to ingest or apply their medication. The lack of medication treatment on a particular day or timeframe may be detected by the system through measurement of sleep, breathing, SpO2 and other parameters. When the system report indicates a change inconsistent with well-treated sleep, the report may notify the user (after waking), a medical advocate, a doctor, and other persons.

In some examples, embodiments may report on non-compliance of its own elements, for example when the user applies a stimulation patch and sleeps expecting electrical stimulation but the battery or other element of the stimulation patch fails and the electrical stimulation regime is not performed. A similar example may be the failure of the wireless connection from smart controller to patch.

In some examples, the recording of compliance may be written into memory on one or more of the patches. In some examples, the recording of compliance may be written into memory on the smart controller. In some examples, the recording of compliance may be written into memory on a server, such as in the cloud.

### Optimized Hypoxia Handling

Embodiments use hypoxia to treat various conditions. Hypoxia is the condition of lowered blood oxygen levels. Sleep apnea causes hypoxia. The hypoxia severity, or hypoxic burden, is calculated from the inter-episode interval, the durations of the individual hypoxia events and the sum of the time spent in hypoxia. As a hypoxia event lasts longer, the effects on the person are increased and the hypoxic burden increases.

Intermittent Hypoxia (“IH”) is characteristic of sleep apnea and has some beneficial effects due to resilience in the body's systems, including the nerves and muscles, to accommodate adverse conditions. However, there is a threshold of occurrence and severity, which varies by individual and by scenario, above which the beneficial effects of IH become detrimental effects. Chronic Intermittent Hypoxia (“CIH”) can range in severity from beneficial levels to harmful levels, with a transition from beneficial to detrimental level termed a threshold that varies by individual. The process of using hypoxia below this threshold, generally up to 10-14% O2 deficiency at rest interspersed with intervals of normoxia or hyperoxia, is termed intermittent hypoxic conditioning (“IHC”).

Examples of the beneficial effect of intermittent hypoxia when kept below the threshold limit are improved respiratory and non-respiratory somatic nervous function, and increased growth/trophic factor expressions in the central nervous system which improve neuroplasticity and neuroprotection.

Clinical studies have shown that mild intermittent hypoxia improves respiratory plasticity and strengthens the upper airway, thereby strengthening the upper airway and reducing the need for sleep apnea intervention such as CPAP.

Embodiments are able to operate coincidentally with a measurement of hypoxic burden in real time during the sleep period. When the detection and treatment of OSA episodes causes the hypoxic burden to be reduced and fall into the beneficial zone for IHC, then treatment to eliminate sleep apnea events is adjusted such that the hypoxic burden remains in the beneficial zone.

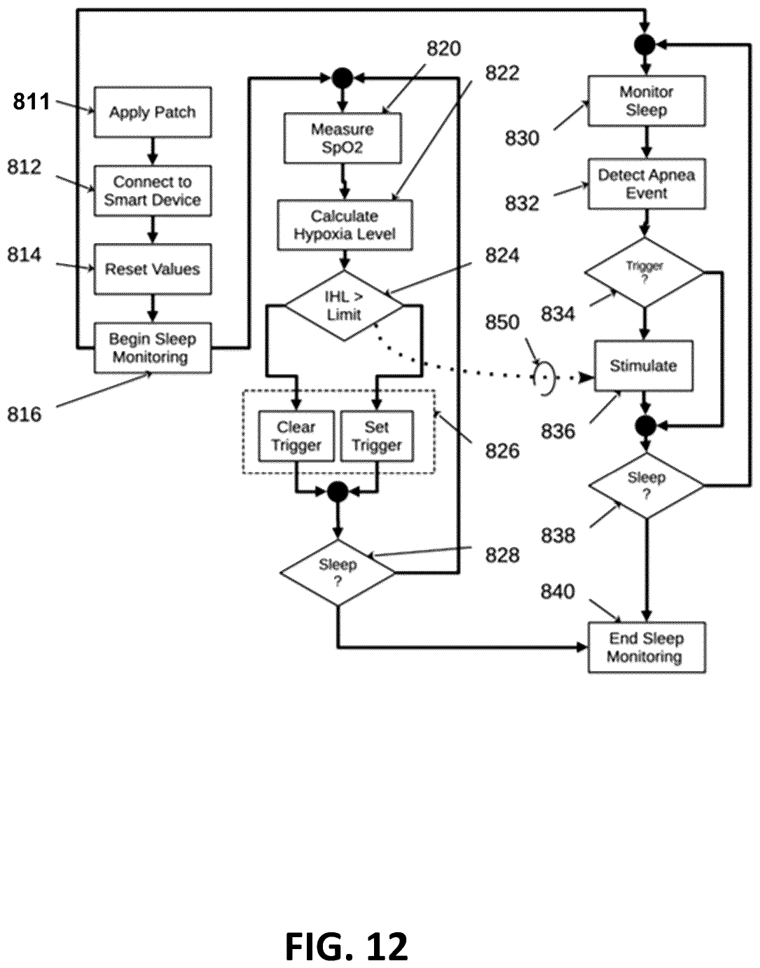

FIG. 12 is a flow diagram of the functionality of Obstructive Sleep Apnea Detection and Stimulation System 100 when monitoring sleep while treating apnea events and modifying the hypoxia level in accordance to embodiments. In one embodiment, the functionality of the flow diagram of FIG. 12 is implemented by software stored in memory or other computer readable or tangible medium, and executed by a processor. In other embodiments, the functionality may be performed by hardware (e.g., through the use of an application specific integrated circuit (“ASIC”), a programmable gate array (“PGA”), a field programmable gate array (“FPGA”), etc.), or any combination of hardware and software.

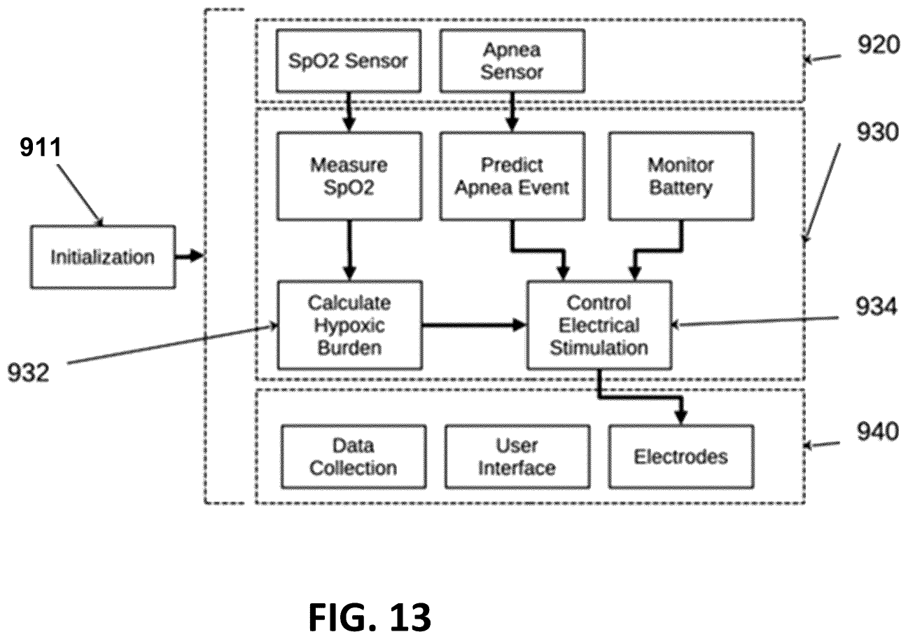

FIG. 13 is a block diagram of the elements of Obstructive Sleep Apnea Detection and Stimulation System 100 in accordance to embodiments.

In embodiments, patch 110 includes one or more sensors to measure the blood oxygen level of the user in real time. Inasmuch as hypoxic burden is a function not only of frequency of hypoxic events, but also of their duration, a Hypoxic Calculator 920 combines the period measurements in the time domain from one event's onset to the next with an integration of each event's magnitude. The Hypoxic Calculator compares the measured SpO2 with a triggering level. Whenever the measured value is less than the triggering level, the event's magnitude is increased.

In some examples, the integration of event magnitude is linear as the SpO2 level falls below the triggering level.

In some examples, the integration of event magnitude is non-linear as the SpO2 falls more and more below the triggering level, reflecting the increasingly large effect of hypoxia at lower levels of SpO2.

Referring to FIG. 12, the one or more patches 110 is/are applied at 811 and the one or more patches then connect to the Smart Controller 140 at 812. The Smart Controller resets the calculated values 814, including the real-time calculated value for hypoxic burden. The user's sleep then begins to be monitored at 816. Two processes proceed in parallel: monitoring hypoxia by measuring SpO2 at 820, and monitoring apnea by monitoring sleep at 830. A running hypoxia level is calculated at 822 and a Trigger is set or cleared at 826 according to whether the hypoxia level is above or below the limit. Sleep continues being monitored at 828. While monitoring sleep at 830, each detected apnea event at 832 is then processed according to whether the Trigger is set or cleared. If the Trigger is set, then the user is stimulated in order to eliminate the apnea event. If the Trigger is not set, then the user is not stimulated, allowing the apnea event to affect the hypoxia level. Sleep continues to be monitored 838. When the system detects a waking state, sleep monitoring ends at 840. The level of hypoxic burden affects the decision to stimulate the user at an apnea event at 850.

Referring to FIG. 13, system 100 includes Initialization 911, Sensor and Peripheral Management 920, Data Calculation 930, and Output Management 940. Data Calculation includes the Hypoxic Calculator 932 and the Electrical Stimulation Controller 934. Output Management includes data collection, the user interface and the electrical stimulation through the electrodes.

In general, an apnea event causes a delayed drop in SpO2. A sequence of hypopnea events causes a slow drop in SpO2. The Hypoxic Calculator 932 considers the history of hypoxic events in real time to adjust the magnitude of the hypoxic burden on the user. For example, a sequence of hypopnea events, each lasting less than 10 seconds and of mild severity may affect the total hypoxic burden more or less than two more widely-separated sleep apnea events, each of longer duration and more serious SpO2 severity.

In some examples, patch 110 monitors other conditions during sleep using sensors, such as sleep position, in order to optimize the handling of apnea events since the impact of a series of such events may be different during supine sleep compared to side-sleeping or prostrate sleeping.

The features, structures, or characteristics of the disclosure described throughout this specification may be combined in any suitable manner in one or more embodiments. For example, the usage of “one embodiment,” “some embodiments,” “certain embodiment,” “certain embodiments,” or other similar language, throughout this specification refers to the fact that a particular feature, structure, or characteristic described in connection with the embodiment may be included in at least one embodiment of the present disclosure. Thus, appearances of the phrases “one embodiment,” “some embodiments,” “a certain embodiment,” “certain embodiments,” or other similar language, throughout this specification do not necessarily all refer to the same group of embodiments, and the described features, structures, or characteristics may be combined in any suitable manner in one or more embodiments.

One having ordinary skill in the art will readily understand that the embodiments as discussed above may be practiced with steps in a different order, and/or with elements in configurations that are different than those which are disclosed. Therefore, although this disclosure considers the outlined embodiments, it would be apparent to those of skill in the art that certain modifications, variations, and alternative constructions would be apparent, while remaining within the spirit and scope of this disclosure. In order to determine the metes and bounds of the disclosure, therefore, reference should be made to the appended claims.

What is claimed is:

1. A method of treatment for obstructive sleep apnea (OSA) of a user, the method comprising:
affixing a patch externally on a dermis of the user, the patch comprising a flexible substrate, a processor coupled to the substrate, and an array of electrodes coupled to the substrate, wherein the array of electrodes comprises a plurality of electrode pairs in different geometric positions on the substrate;
detecting an occurrence of OSA;
in response to the detecting, generating a first electrical stimuli to one or more nerves or tissues of the user via a first selected subset of pairs of the plurality of electrode pairs; and
generating a second electrical stimuli to one or more nerves or tissues of the user via a second selected subset of pairs of the plurality of electrode pairs sequentially after generating the first electrical stimuli, wherein the first selected subset of pairs are different than the second selected subset of pairs.

2. The method of claim 1, further comprising:
determining which of the first electrical stimuli or the second electrical stimuli had a greater effect in opening an airway of the user.

3. The method of claim 2, wherein determining when an electrical stimuli has a greater effect in opening the airway of the user corresponds to an amount an electrical stimuli that causes an involuntary response of a tongue of the user.

4. The method of claim 1, wherein the first electrical stimuli is adapted to stimulate a hypoglossal nerve of the user.

5. The method of claim 1, wherein the first electrical stimuli comprises a first timing, a first amplitude and a first duration, and the second electrical stimuli comprises a second timing, a second amplitude and a second duration;
wherein at least one of the first timing, the first amplitude and the first duration is different than the second timing, the second amplitude and the second duration.

6. The method of claim 2, wherein the generating the first electrical stimuli, the generating the second electrical stimuli and the determining which of the first electrical stimuli or the second electrical stimuli had the greater effect all occur while the user is sleeping.

7. The method of claim 1, wherein the first electrical stimuli comprises square waves having an amplitude between 10 and 100 volts, pulse widths between 100 and 500 microseconds, and a pulse repetition rate of between 2 and 60 pulses per second.

8. The method of claim 1, wherein the electrical stimuli comprises a series of pulses with a pattern comprising an intensity and a duration, an applied frequency of the pulses is 2 Hz-60 Hz, and an applied current is 0.1-100 mA.

9. The method of claim 1, wherein the patch is affixed to one of a neck of the user or a jaw of the user.

10. The method of claim 1, wherein the detecting an occurrence of OSA comprises using a machine learning model and input parameters to predict the occurrence.

11. The method of claim 2, further comprising selecting the electrical stimuli that had the greater effect for subsequent electrical stimuli.

12. The method of claim 11, further comprising repeating generating electrical stimuli with different subsets of pairs of the plurality of electrode pairs during a sleep period of the user and repeating the selecting the electrical stimuli that had the greater effect for subsequent electrical stimuli.

13. A obstructive sleep apnea (OSA) treatment system comprising:
a patch adapted to be externally applied on a dermis of a user, the patch comprising a flexible substrate, a processor coupled to the substrate, and an array of electrodes coupled to the substrate, wherein the array of electrodes comprises a plurality of electrode pairs in different geometric positions on the substrate;
the processor, in response to detecting an occurrence of OSA, configured to:
generate a first electrical stimuli to one or more nerves or tissues of the user via a first selected subset of pairs of the plurality of electrode pairs; and
generate a second electrical stimuli to one or more nerves or tissues of the user via a second selected subset of pairs of the plurality of electrode pairs sequentially after generating the first electrical stimuli, wherein the first selected subset of pairs are different than the second selected subset of pairs.

14. The system of claim 13, the processor further configured to:
determine which of the first electrical stimuli or the second electrical stimuli had a greater effect in opening an airway of the user.

15. The system of claim 14, wherein determining when an electrical stimuli has a greater effect in opening the airway of the user corresponds to an amount an electrical stimuli that causes an involuntary response of a tongue of the user.

16. The system of claim 13, wherein the first electrical stimuli is adapted to stimulate a hypoglossal nerve of the user.

17. The system of claim 13, wherein the first electrical stimuli comprises a first timing, a first amplitude and a first duration, and the second electrical stimuli comprises a second timing, a second amplitude and a second duration;
wherein at least one of the first timing, the first amplitude and the first duration is different than the second timing, the second amplitude and the second duration.

18. The system of claim 14, wherein the generating the first electrical stimuli, the generating the second electrical stimuli and the determining which of the first electrical stimuli or the second electrical stimuli had the greater effect all occur while the user is sleeping.

19. The system of claim 14, the processor further configured to select the electrical stimuli that had the greater effect for subsequent electrical stimuli.

20. The system of claim 13, the processor further configured to repeat generating electrical stimuli with different subsets of pairs of the plurality of electrode pairs during a sleep period of the user and repeating the selecting the electrical stimuli that had the greater effect for subsequent electrical stimuli.
